(19) United States
(12) Patent Application Publication
(10) Pub. No.: US 2008/0293939 A1
(43) Pub. Date: Nov. 27, 2008

(54) QUINAZOLINONE DERIVATIVES USEFUL AS ANTI-HYPERALGESIC AGENTS

(76) Inventors: Andrew James Culshaw, London (GB); Edward Karol Dziadulewicz, London (GB); Allan Hallett, London (GB); Terance William Hart, London (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/950,079

(22) Filed: Dec. 4, 2007

Related U.S. Application Data
(63) Continuation of application No. 10/530,897, filed on Mar. 9, 2006, now abandoned.

(30) Foreign Application Priority Data
Oct. 11, 2002 (GB) 0223730.3

(51) Int. Cl. C07D 239/88

(52) U.S. Cl. 544/287

(57) ABSTRACT

The present invention relates to quinazolinones of formula (I) wherein R¹ is hal; a); b); or c); X is N or CR⁸; R² is hal; nitro; C₁-C₆alkylcarbonyl; C₁-C₆alkyl or C₃-C₆cycloalkyl; R³ is C₁-C₆alkyl; C₁-C₆alkoxy or amino; and wherein the further radicals have the meanings as defined in the specification, which compounds exhibit human vanilloid antagonistic activity; to processes for their production, their use as pharmaceuticals and to pharmaceutical compositions comprising them.

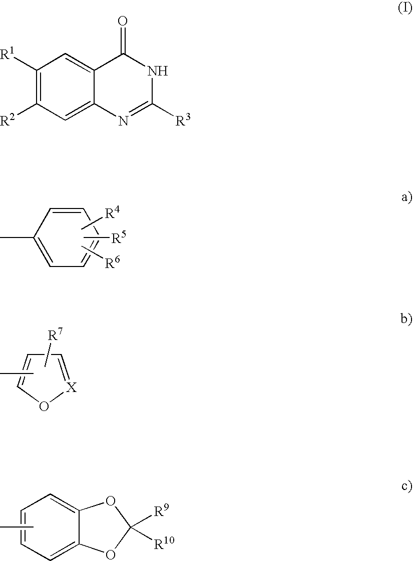

The present invention relates to novel quinazolinone derivatives, to processes for their production, their use as pharmaceuticals and to pharmaceutical compositions comprising them.

More particularly the present invention provides, in a first aspect, a quinazolinone of formula I

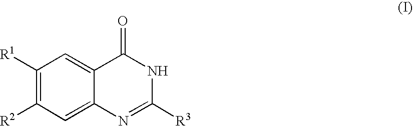

wherein
R² is hal;

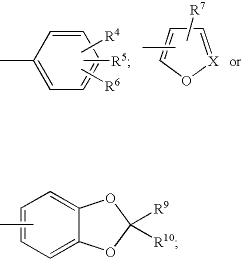

### X is N or CR⁸;

R² is hal; nitro; C₁-C₆alkylcarbonyl; C₁-C₆alkyl or C₃-C₆cycloalkyl;
R³ is C₁-C₆alkyl; C₁-C₆alkoxy or amino;
R⁴ is H; hal; hydroxy; amino; C₁-C₆alkyl-amino, di(C₁-C₆alkyl)-amino, C₁-C₆alkyl; C₁-C₆alkoxy which is unsubstituted or mono-, di- or trisubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C₇cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy,

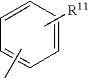

or —O—[CH₂]ₙ-A wherein A represents

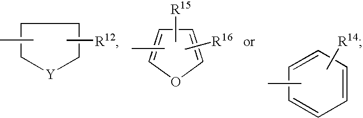

Y represents O or NR¹³,

and n is 0, 1, 2, 3, 4, 5 or 6;

R⁵ and R⁶, independently, are H; hal; C₁-C₆alkoxy; or C₁-C₆alkyl;
R⁷ and R⁸, independently, are H or C₁-C₆alkyl;
R⁹ and R¹⁰, independently, are H or hal;
R¹¹ is H; hal; C₁-C₆alkoxy; or C₁-C₆alkyl;
R¹² is H; hal; C₁-C₆alkoxy; or C₁-C₆alkyl;
R¹³ is H or C₁-C₆alkyl;
R¹⁴ is H; hal; C₁-C₆alkoxy; or C₁-C₆alkyl; and
R¹⁵ and R¹⁶, independently, are H; hal; or C₁-C₆alkyl;
in free base or acid addition salt form.

Terms used in this specification have the following meanings:

“C₁-C₆alkyl” denotes straight chain or branched C₁ to C₆-alkyl, e.g. methyl, ethyl, n-propyl, isopropyl, n-butyl, isobutyl, sec-butyl, tert-butyl.

“C₁-C₆alkoxy” denotes straight chain or branched C₁ to C₆-alkyl-oxy, e.g. methoxy, ethoxy, n-propoxy or isopropoxy.

“Hal” denotes halogen which may be I, Br, Cl or F.

Compounds of the invention exist in free or salt, e.g. acid addition salt form. The invention is to be understood as including the compounds of formula I in free as well as in salt form, e.g. as trifluoroacetate or hydrochloride salt. Suitable pharmaceutically acceptable acid addition salts for pharmaceutical use in accordance with the invention include in particular the hydrochloride salt.

In formula I the following significances are preferred independently, collectively or in any combination or sub-combination:

### (a) R¹ is

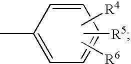

(b) R² is isopropyl;
(c) R³ is methyl or amino; more preferably methyl;
(d) R⁴ is in meta position as defined above; or more preferably R⁴ is in meta position and is C₁-C₄alkoxy or C₃-C₄cycloalkylC₁-C₄alkoxy;
(e) R⁵ is in para position and is hal; more preferably Cl;
(f) R⁶ is H or halogen; more preferably H;
(g) R⁷ or R⁸ is H or methyl; more preferably methyl;
(h) R¹⁴ is C₁-C₄alkoxy; more preferably methoxy;
(i) R¹² is methyl;
(k) R¹³ is methyl;

### (I) n is 0 or 1 or 2; and

(m) R⁹ and R¹⁰ are hydrogen or fluoro.

In addition to the foregoing the present invention also provides a process for the production of a compound of formula I which process comprises coupling suitable starting compounds applying methods known to the skilled artisan.

More particularly the invention provides a process for the production of a compound of formula I comprising the steps of:

a) for the production of a compound of formula I wherein R³ is not NH₂, reacting a compound of formula II

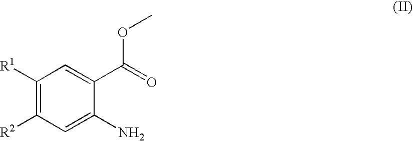

wherein
R² and R² are as defined in formula I,
with a compound of formula III

N≡C—R³ (III)

wherein R³ is as defined in formula I in the presence of an acid, e.g. hydrogen chloride; or

b) for the production of a compound of formula I wherein R³ is NH₂, reacting a compound of formula IV

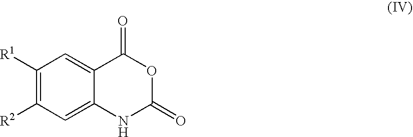

wherein R² and R² are as defined in formula I,
with 2-ethyl-2-thiopseudourea hydrobromide;
and recovering the obtained compound in free or in salt form, e.g. acid addition salt form.

Compounds of formula I resulting from the above process may be further derivatised, e.g. as described in Example 1, i.e. for the conversion of 6-(4-chloro-3-fluoro-phenyl)-7-isopropyl-2-methyl-3H-quinazolin-4-one into 6-(4-chloro-3-cyclopropylmethoxy-phenyl)-7-isopropyl-2-methyl-3H-quinazolin-4-one.

Compounds of formula III are known or may be prepared from corresponding known compounds, e.g. as described in Examples 1 or 2. Compounds of formula IV are known or may be prepared from corresponding known compounds, e.g. as described in Example 59.

Compounds of formula II are new and constitute part of the present invention. They may be prepared from corresponding known starting materials according to the general knowledge of a person skilled in the art, e.g. as described in Examples 1 and 2. For instance, an acid of formula V

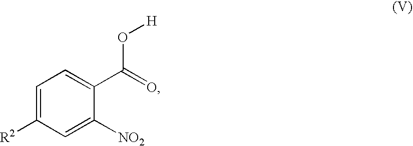

wherein R² has the meaning as provided above for a compound of formula I, is transformed into an ester in a manner known as such to provide a nitro compound of formula VI

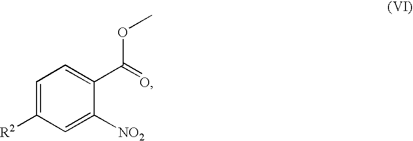

wherein R² has the meaning as provided above for a compound of formula I. The nitro compound of formula VI is then reduced to the corresponding amine of formula VII

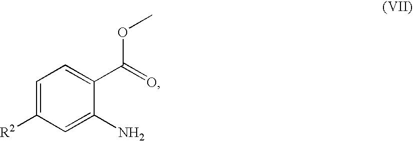

wherein R² has the meaning as provided above for a compound of formula I, e.g. by reaction with hydrogen in the presence of a suitable catalyst, such as palladium on activated carbon. The obtained amine of formula VII can be further reacted to the iodide of formula VIII,

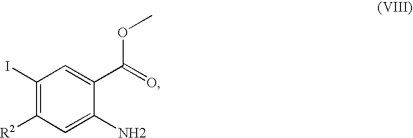

wherein R² has the meaning as provided above for a compound of formula I, e.g. by reaction firstly with silver (I) sulfate and secondly with iodide in a suitable solvent.

The obtained iodide of formula VII can be reacted with the boronic acid of formula IX

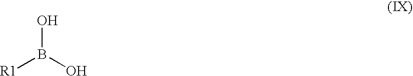

wherein R² has the meaning as provided above for a compound of formula I, providing a compound of formula II.

Working up the reaction mixtures according to the above processes and purification of the compounds thus obtained may be carried out in accordance to known procedures.

Acid addition salts may be produced from the free bases in known manner, and vice-versa.

Compounds of formula I in optically pure form can be obtained from the corresponding racemates according to well-known procedures, e.g. HPLC with chiral matrix. Alternatively, optically pure starting materials can be used.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

In the additional process steps, carried out as desired, functional groups of the starting compounds which should not take part in the reaction may be present in unprotected form or may be protected for example by one or more of the protecting groups mentioned below. The protecting groups are then wholly or partly removed according to one of the methods described there.

The protecting groups may already be present in precursors and should protect the functional groups concerned against unwanted secondary reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of such functional groups by protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

All process steps described here can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralising agents, for example ion exchangers, typically cation exchangers, for example in the H+ form, depending on the type of reaction and/or reactants at reduced, normal, or elevated temperature, for example in the range from −100° C. to about 190° C., preferably from about −80° C. to about 150° C., for example at −80 to −60° C., at room temperature, at −20 to 40° C. or at the boiling point of the solvent used, under atmospheric pressure or in a closed vessel, where appropriate under pressure, and/or in an inert atmosphere, for example under argon or nitrogen.

The compounds of formula I and their pharmaceutically acceptable acid addition salts (hereinafter: the agents of the invention) have beneficial pharmacological activity and are useful as pharmaceuticals. In particular the agent of the invention exhibit human vanilloid antagonistic activity. More particularly, the agents of the invention, e.g. the compounds of examples 1-60 are active, e.g. at the human vanilloid receptor type 1 (VR1).

Vanilloid receptor interaction of the agents of invention is demonstrated by the following test 1.

### Test I: Fluorescence Assay

Cultures of Chinese Hamster Ovary (CHO) cells expressing human VR1 ion channels are prepared according to standard protocols [McIntyre et al., British Journal of Pharmacology 132: 1084-1094 (2001)]. The activity of test compounds are investigated using a fluorescence assay utilising calcium sensitive dyes to measure changes in intracellular calcium ion concentration. The cells are plated at a density of 40,000 per well on 96 well Costar black, clear bottomed plates cultured at 37° C. in 5% CO₂ in MEM medium overnight. On the day of the assay, cells are incubated in 2 μM fura-2/AM (Molecular Probes) made up in assay buffer (Hank's Balanced Salt Solution (HBSS, Invitrogen) containing 10 mM N-2-(hydroxyethylpiperazine-N′-[2-ethanesulfonic acid) (HEPES), pH 7.4] containing 0.01% pluronic F-127 for 40 min at room temperature. After washing twice with assay buffer, 100 μl assay buffer, or test compounds (range from 1 nM to 10 μM final) where appropriate, are added to each well and the plate incubated for ten minutes at room temperature and then placed in a Molecular Devices Flexstation. The fluorescence is measured over 1 min at 4s intervals using excitation wavelengths of 340 and 380 nm and emission of 520 nm. Human vanilloid receptor 1 ion channels are stimulated by application of either the agonist capsaicin or low pH. At approximately 17s, 20 μl of capsaicin made up at 6 fold the required final concentration were transferred to the cells. For pH experiments, 100 μl HBSS alone pH 7.4 (containing test compounds) is added to the cells and 20 μl of 60 mM 2-[N-morpholino]ethane sulfonic acid (MES) in HBSS transferred to the cells. The pH of this solution is adjusted such that it gives the desired pH when diluted 1:6. The ratio of fluorescence intensities following excitation at 340 and 380 nm is calculated for each time point. The agonist-evoked response is calculated as the mean of the ratios in the four time-points following stimulation minus the basal ratio.

In the above test the agents of the invention effectively block Ca-uptake in the range from about 1 nM to about 10 μM, especially 25 to 100 nM, especially 50 or 60 nM.

In view of the above, the agent of the invention are useful in the prevention and treatment of diseases and conditions in which human VR1 activation plays a role or is implicated. Such conditions include in particular chronic pain, i.e. for the treatment of hyperalgesia and, in particular, for the treatment of severe chronic pain; neuropathic pain associated with post-herpetic neuralgia, amputations (“phantom limb pain”), reflex sympathetic dystrophy and other chronic nerve injuries; inflammatory pain, e.g. chronic inflammatory pain, bone and joint pain (osteoarthritis), cancer pain, myofascial pain (muscular injury, fibromyalgia) and perioperative pain (general surgery, e.g. associated with burns, sprains, fracture or the like, subsequent to surgical intervention, gynecologic surgery); or in asthma, for example, aluminosis, anthracosis, inflammatory diseases for example inflammatory airways disease, e.g. Chronic Obstructive Pulmonary Disease; asbestosis, chalicosis, ptilosis, siderosis, silicosis, tabacosis, byssinosis, and rhinitis; smooth muscle relaxants, e.g. for the treatment of spasm of the gastro-intestinal tract or uterus, e.g. in the therapy of Crohn's disease, ulcerative colitis or pancreatitis, inflammatory bowel disease, cystitis, e.g. interstitial cystitis, pancreatitis, and uveitis; inflammatory skin disorders and rheumatoid arthritis, inflammatory skin disorders, for example psoriasis and eczema.

Activity specifically as analgesic agents may be demonstrated in accordance with standard test methods, e.g. as described in the following test 2.

### Test 2: Anti-hyperalgesic Effects in a Model of Neuropathic Pain in the Rat

Peripheral neuropathy is induced by partial ligation of the left sciatic nerve. Mechanical hyperalgesia is assessed from paw withdrawal thresholds measured on the ipsilateral (ligated) and contralateral (non-ligated) hindpaws using standard paw pressure methods. Drug effects are studied 11-15 days post ligation. The mean paw withdrawal threshold: s.e.m. for the left (ligated) paw is compared to that of the right (non-ligated) paw. Pharmaceutical Compound is administered, e.g. orally in 20% cremophor/water in a volume of 1 ml. The post-drug percentage hyperalgesia values are obtained by comparison to the pre-drug value for the right (non-ligated) paw; this enables a true measure of the reduction in hyperalgesia to be obtained without the added complication of any drug effects on the right paw. Single oral administration of Pharmaceutical Compound produces a highly effective reversal of mechanical hyperalgesia in the partially denervated rat hind paw. Pharmaceutical Compounds produce a reversal of mechanical hyperalgesia at 30 mg/kg and show a rapid onset of activity with a long duration of action. Thus, Pharmaceutical Compounds are potent and efficacious anti-hyperalgesic agents following oral administration in a rat model of neuropathic pain.

Preferred are quinazolinones of formula I wherein

R¹ is hal;

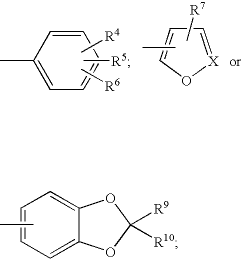

### X is N or CR⁸;

R² is C₁-C₆alkyl;
R³ is C₁-C₆alkyl; C₁-C₆alkoxy or amino;
R⁴ is H; hal; hydroxy; amino; C₁-C₆alkyl-amino, di(C₁-C₆alkyl)-amino, C₁-C₆alkyl; C₁-C₆alkoxy which is unsubstituted or mono-, di- or trisubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C₇cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (CI-C₆alkyl)₂N-C₁-C₆alkoxy; C₁-C₆alkyl-sulfanyl; C₁-C₆alkyl-sulfanylC₁-C₆alkoxy,

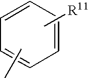

or —O—[CH₂],ₙ-A wherein A represents

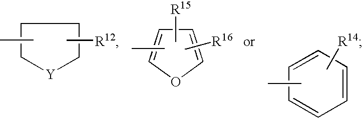

Y represents O or NR¹³,
and n is 0, 1, 2, 3, 4, 5 or6;
R⁵ and R⁶, independently, are H; hal; C₁-C6alkoxy; or C₁-C₆alkyl;
R⁷ and R⁸, independently, are H or C₁-C₆alkyl;
R⁹ and R¹⁰, independently, are H or hal;
R¹¹ is H; hal; C₁-C₆alkoxy; or C₁-C₆alkyl;
R¹² is H; hal; C₁-C₆alkoxy; or C₁-C₆alkyl;
R¹³ is H or C₁-C₆alkyl;
R¹⁴ is H; hal; C₁-C₆alkoxy; or C₁-C₆alkyl; and
R¹⁵ and R¹⁶, independently, are H; hal; or C₁-C₆alkyl.

Very preferred are those quinazolinones of formula I wherein
R¹ is hal;

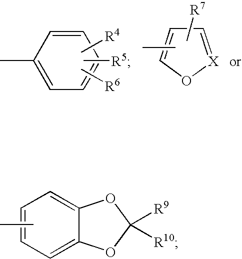

X is N or CR⁸;
R² is C₁-C₆alkyl;
R³ is C₁-C₆alkyl or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, or —O—[CH₂]ₙ-A wherein A represents

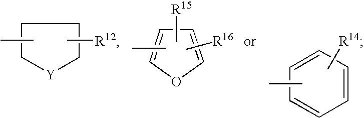

Y represents O or NR¹³,
and n is 0, 1 or 2;
R⁵ and R⁶, independently, are H; hal; or C₁-C₆alkoxy;
R⁷ and R⁸, independently, are H or C₁-C₆alkyl;
R⁹ and R²⁰, independently, are H or hal;
R¹² is H;
R¹³ is C₁-C₆alkyl;
R¹⁴ is H; or C₁-C₆alkoxy; and
R¹⁵ and R¹⁶ are H.

Even more preferred are quinazolinones of formula I wherein
R¹ is hal;

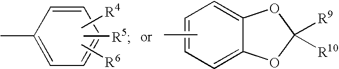

R² is hal; nitro; C₁-C₆alkylcarbonyl; C₁-C₆alkyl or C₃-C₆cycloalkyl;
R³ is C₁-C₆alkyl; C₁-C₆alkoxy or amino;
R⁴ is H; hal; hydroxy; C₁-C₆alkyl; C₁-C₆alkoxy; C₁-C₆alkoxyC,-C₆alkoxy; C₁-C₆alkoxyC₁-C₆alkoxyC₁-C₆alkoxy; C₁-C₆alkoxyC₁-C₆alkyl; halogenoC₁-C₆alkoxy; C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy,

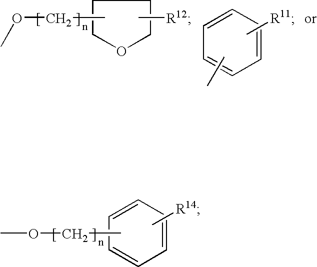

wherein n is 0, 1, 2, 3, 4, 5 or 6;
R⁵, R⁶, R¹¹ and R¹⁴, independently, are H; hal; C₁-C₆alkoxy: or C₁-C₆alkyl;
R¹² is H or C₁-C₆alkyl; and
R⁹ and R¹⁰, independently, are H or hal;
in free base or acid addition salt form.

Most preferred are those compounds disclosed in the Examples below.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.05 to about 150, preferably from about 0.1 to about 100 mg/kg animal body weight. In larger mammals, for example humans, an indicated daily dosage is in the range from about 0.5 to about 5000, preferably from about 1 to about 500mg of an agent of the invention, conveniently administered, for example, in divided doses up to four times a day or in sustained release form.

The agents of the invention can be administered in vivo either alone or in combination with other pharmaceutical agents, e.g. agents effective in the treatment of diseases and conditions in which the human VR1 activation plays a role or is implicated including cyclooxygenase-2 (COX-2) inhibitors, such as specific COX-2 inhibitors (e.g. celecoxib, COX189, and rofecoxib) or in general nonsteroidal anti-inflammatory drugs (NSAIDs) (e.g. acetylsalicylic acid, propionic acid derivatives), tricyclic antidepressants (e.g. Anafranil®, Asendin®, Aventyl®, Elavil®, Endep®, Norfranil®, Norpramin®, Pamelor®, Sinequan®, Surmontil®, Tipramine®, Tofranil®, Vivactil®, Tofranil-PM®), anticonvulsants (e.g. gabapentin), GABA_{B} agonists (e.g. L-baclofen), opioids and CB receptor agonists, e.g. CB₁ receptor agonists.

The pharmaceutical compositions for separate administration of the combination partners and for the administration in a fixed combination, i.e. a single galenical composition comprising at least two combination partners, according to the invention can be prepared in a manner known per se and are thus suitable for enteral, such as oral or rectal, and parenteral administration to mammals, including man, comprising a therapeutically effective amount of at least one pharmacologically active combination partner alone or in combination with one or more pharmaceutically acceptable carriers, especially suitable for enteral or parenteral application.

Pharmaceutical compositions contain, for example, from about 0.1% to about 99.9%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

Moreover the present invention provides the use of an agent of the invention, for the manufacture of a medicament for the treatment of any condition mentioned above.

In still a further aspect the present invention provides a method for the treatment of any condition mentioned above, in a subject in need of such treatment, which comprises administering to such subject a therapeutically effective amount of an agent of the invention.

The following examples illustrate the invention.

### ABBREVIATIONS

conc. concentrated
DCM dichloromethane
DMF dimethyl formamide
DMSO dimethyl sulfoxide
EtOAc ethyl acetate
HPLC high pressure liquid chromatography
Me methyl
mp melting point
MS mass spectrometry
NMR nuclear magnetic resonance
THF tetrahydrofuran

### EXAMPLE 1

### Preparation of 6-(4-Chloro3-cyclopropylmethoxy-phenyl)7-isopropyl-2-methyl-3H-quinazolin-4-one

a) Preparation of 4-Isopropyl-2-nitro-benzoic acid: A stirred solution of 2-nitro-4-cymene (8 g, 0.0446 mol) in t-butoxy bis(dimethylamino)methane (10 g 0.0574 mol) is heated at 110° C. for 10 h. The deep red solution is cooled to room temperature and the excess reagent and by-products are removed under reduced pressure. The residue is dissolved in tert. butanol (600 ml) and a solution of potassium acetate (51.35 g, 0.372 mol) in water (150 ml) is added. Potassium permanganate (51.35 g, 0.325 mol) is added portion-wise to this mixture producing a slight exotherm. After 3 h, the mixture is filtered through celite and the celite pad washed with water (500 ml) and methanol (1000 ml). The volatiles are evaporated under reduced pressure and the residue partitioned between ethyl acetate and water. The aqueous layer is acidified to pH3 using hydrochloric acid and the mixture is extracted with ethyl acetate (3×150 ml). The combined ethyl acetate extracts are washed with saturated brine, dried (MgSO₄), filtered and evaporated under reduced pressure to give 4-Isopropyl-2-nitro-benzoic acid as a brown solid. This is sufficiently pure for use in the next step without further purification. ¹H NMR (CDCl₃, 400 MHz) δH (ppm) 10.0 (1H. br s), 7.82 (1H, d, J=8.0 Hz), 7.64 (1H, d, J=1.5 Hz), 7.50 (1H, dd, J=1.5,8.0 Hz), 3.05 (1H, m), 1.30 (6H, d, J=6.9 Hz).

b) Preparation of 4-Isopropyl-2-nitro-benzoic acid methyl ester: To a stirred solution of 4-Isopropyl-2-nitro-benzoic acid (6.7 g, 0.032 mol) in dry DMF (100 ml) at room temperature is added cesium carbonate (16.0 g, 0.049 mol). After 30 minutes, iodomethane (6.84 g, 0.048 mol) is added and the mixture is stirred at room temperature for 16 h. The mixture is poured into water (500 ml) and extracted with ethyl acetate (3×100 ml). The combined EtOAc extracts are washed with water (200 ml), saturated brine (100 ml), dried (MgSO₄), filtered and evaporated to give a red oil. Purification by column chromatography on silica gel using cyclohexane/ethyl acetate (10:1) as eluant gave 4-isopropyl-2-nitro-benzoic acid methyl ester

c) Preparation of 2-Amino-4-isopropyl-benzoic acid methyl ester: To a stirred solution of 4-isopropyl-2-nitro-benzoic acid methyl ester (6.0 g, 0.027 mol) in methanol (200 ml) at room temperature under argon is added 10% palladium on activated carbon (5.4 g). The suspension is evacuated and purged with hydrogen three times and then stirred at room temperature for 18 h. The reaction is then placed under argon atmosphere and filtered through a pad of celite. The celite pad is washed with ethyl acetate and the filtrate and washings evaporated under reduced pressure to give a colourless oil. Purification by column chromatography on silica gel using cyclohexanelethyl acetate (10:1) as eluant gave 2-amino-4-isopropyl-benzoic acid methyl ester.

d) Preparation of 2-Amino-5-iodo-4-isopropyl-benzoic acid methyl ester: To a stirred solution of 2-amino-4-isopropyl-benzoic acid methyl ester (4.73 g, 0.0245 mol) in ethanol (100 ml) at room temperature is added silver (I) sulfate (7.64 g, 0.0245 mol). A solution of iodine (6.23 g, 0.0245 mol) in ethanol (200 ml) is added via a pressure-equalised dropping funnel at room temperature and the mixture is then stirred at room temperature for 1 h. After filtration of the crude reaction mixture through a pad of celite, the ethanol is evaporated and the residue partitioned between water/ethyl acetate and extracted with ethyl acetate (3×100 ml). The ethyl acetate extracts are combined and washed with saturated brine, dried (MgSO₄), filtered and evaporated to give a red solid. The crude product could be used directly or purified by chromatography on silica gel using cyclohexane/ethyl acetate (10:1) as eluant followed by recrystallisation from hexanes to give 2-Amino-5-iodo-4-isopropyl-benzoic acid methyl ester. ¹H NMR (CDCl₃, 400 MHz) δH (ppm) 8.25 (1H, s), 6.55 (1H, s), 5.69 (2H, br s), 3.85 (3H, s), 3.06 (1H, m), 1.19 (6H, d, J=6.8 Hz).

e) Preparation of 4-Chloro-3-fluorobenzeneboronic acid: A stirred solution of 4-bromo-1-chloro-2-fluorobenzene (25 g, 0.119 mol) and triisopropylborate (30.5 ml, 0.131 mol) in dry THF (500 ml) under argon is cooled to −100° C. and n-butyllithium (52.5 ml of a 2.5M solution in hexanes, 0.131 mol) is added dropwise over 15 min. The reaction mixture is allowed to warm gradually to room temperature over 18 h before it is quenched by the addition of 2M hydrochloric acid (250 ml) and stirred at room temperature overnight. The THF is removed under reduced pressure, the aqueous residue is diluted with water (500 ml) and the mixture is extracted with diethyl ether (3×200 ml). The combined ether extracts are washed with saturated brine (200 ml), dried (MgSO₄), filtered, evaporated and dried in vacuo to give a colourless solid—a mixture of 4-Chloro-3-fluorobenzeneboronic acid and the functionally equivalent cyclotriboroxane.

f) Preparation of 4-Amino-4′-chloro-3′-fluoro-6-isopropyl-biphenyl-3-carboxylic acid methyl ester: To a stirred mixture of 4-chloro-3-fluorobenzeneboronic acid (12.3 g, 0.071 mol), 2-amino-5-iodo-4-isopropyl-benzoic acid methyl ester (18 g, 0.0564 mol) and 1,1′-bis(diphenylphosphino)ferrocenedichloropalladium (II) (1.35 g, 1.65 mmol) in dry DMF (250 ml) under argon is added sodium carbonate (140 ml of a 2M aqueous solution, 0.28 mol). The mixture is heated at 80° C. for 16h, cooled to room temperature and poured into diethyl ether (500 ml). The ether layer is separated, washed with water (3×250 ml) and then saturated brine (50 ml), dried (MgSO₄), filtered and evaporated under reduced pressure to give a brown oil. Purification by column chromatography on silica gel using cyclohexane and then cyclohexane/ethyl acetate (50:1) as eluant gave pure product. The impure product-containing fractions are combined, evaporated and recrystallised from n-hexane with a trace of ethyl acetate to give further 4-Amino-4′-chloro-3′-fluoro-6-isopropyl-biphenyl-3-carboxylic acid methyl ester.

g) Preparation of 6-(4-Chloro-3-fluoro-phenyl)-7-isopropyl-2-methyl-3H-quinazolin-4-one: Hydrogen chloride gas is bubbled through a solution of 4-amino-4′-chloro-3′-fluoro-6-isopropyl-biphenyl-3-carboxylic acid methyl ester (12.6 g, 0.039 mol) in dry acetonitrile (250 ml) for 15 min at room temperature. The bubbling is then stopped and the mixture heated at reflux for 2 h, cooled to room temperature and the volatiles removed under reduced pressure. The colourless residue is poured into water (500 ml) and sodium bicarbonate is added portion-wise until no further CO₂ evolution takes place. The mixture is extracted with dichloromethane (3×200 ml) and the DCM extracts are combined and washed sequentially with water (50 ml) and saturated brine (50 ml), dried (MgSO₄), filtered and concentrated to about 50 ml volume of DCM under reduced pressure. The resulting suspension is filtered, washed with n-hexane and dried to give the title compound as a colourless solid. The filtrate and washings were evaporated to give a beige solid which was sonicated in hexane/DCM, filtered, washed with hexane and dried to give further pure 6-(4-Chloro-3-fluoro-phenyl)-7-isopropyl-2-methyl-3H-quinazolin-4-one.

h) Preparation of 6-(4-Chloro-3-cyclopropylmethoxy-phenyl)-7-isopropyl-2-methyl-3H-quinazolin-4-one: To a stirred solution of 6(4-chloro-3-fluoro-phenyl)-7-isopropyl-2-methyl-3H-quinazolin-4-one (6 g, 0.0185 mol) and cyclopropylcarbinol (7.35 ml, 0.09 mol) in dry N-methylpyrollidinone (75 ml) is added, portionwise, sodium hydride (60% dispersion on mineral oil, 3.6 g, 0.09 mol). When addition is complete, the mixture is heated at 60° C. for 2 h, cooled to room temperature and poured into water (300 ml). The mixture is extracted with cyclohexane (2×100 ml) to remove the mineral oil and then extracted with ethyl acetate (5×100 ml). The ethyl acetate extracts are combined and washed with water (200 ml) and then saturated brine (100 ml), dried (MgSO₄), filtered and evaporated to give a colourless solid. This is recrystallised from ethyl acetate to give 6-(4-Chloro-3-cyclopropylmethoxy-phenyl)-7-isopropyl-2-methyl-3H-quinazolin-4-one after drying. ¹H NMR (CDCl₃, 400 MHz) δH (ppm) 11.51 (1H, br s), 8.06 (1H, s), 7.69 (1H, s), 7.41 (1H, d, J=7.8 Hz), 6.87-6.84 (2H, m), 3.91 (2H, d, J=6.7 Hz), 3.14 (1H, m), 2.57 (3H, s), 1.33 (1H, m), 1.22 (6H, d, J=6.8 Hz), 0.67 (2H, m), 0.39 (2H, m); HPLC RT=6.8 minutes (Phenomenex Luna C18 3 micron column (30×4.6 mm); gradient elution: 10-100% MeCN in water (+0.08% formic acid) over 10 minutes at 3.0 mL/minute); MH+383.

### EXAMPLE 2

### Preparation of 6-(4-Chloro-3-propoxy-phenyl)-7-isopropyl-2-methyl-3H-quinazolin-4-one

a) Preparation of 4-Bromo-1-chloro-2-propoxybenzene: To a stirred solution of n-propanol (10.8 ml, 0.143 mol) in dry DMF (250 ml) at 0° C. is added, portion-wise, sodium hydride (60% dispersion on mineral oil, 5.72 g, 0.143 mol). When addition is complete, the mixture is stirred at 0° C. until effervescence had subsided. The mixture of sodium propoxide thus produced is added to a cooled (0° C.) solution of 4-bromo-1-chloro-2-fluorobenzene (10 g, 0.048 mol) in dry DMF (40 ml) and then allowed to warm to room temperature over 18 h. The volume of DMF is reduced in vacuo and the residue poured into water (500 ml). The mixture is extracted with diethyl ether (3×200 ml) and the ether extracts are combined and washed with water (250 ml) and then saturated brine (100 ml), dried (MgSO₄), filtered and evaporated to give a colourless oil. Purification by column chromatography on silica gel (110 g) using cyclohexane as eluant gave 4-Bromo-1-chloro-2-propoxybenzene.

b) Preparation of 4-Chloro-3-propoxybenzeneboronic acid: A stirred solution of 4-bromo-1-chloro-2-isopropoxybenzene (11.98 g, 0.048 mol) and triisopropylborate (12.26 ml, 0.053 mol) in dry THF (200 ml) under argon is cooled to −78° C. and n-butyllithium (21.1 ml of a 2.5M solution in hexanes, 0.053 mol) is added dropwise. The reaction mixture is allowed to warm gradually to room temperature over 8 h before it is quenched by the addition of 2M hydrochloric acid (100 ml) and stirred at room temperature overnight. Most of the THF is removed under reduced pressure and the mixture is diluted with diethyl ether (500 ml). The ether layer is separated and washed with water (3×200 ml) and then saturated brine (100 ml), dried (MgSO₄), filtered and evaporated to give a colourless solid. This is sonicated with n-hexane, filtered and dried to give 4-Chloro-3-propoxybenzeneboronic acid as a 2:1 mixture with the corresponding cycloboroxane. ¹H NMR (CDCl₃, 400 MHz) 6H (ppm) 7.72-7.68 (2H, m), 7.50 (1H, d, J=7.8 Hz), 7.39 (0.5H, d, J=7.8 Hz), 7.31 (0.5H, br d), 7.19 (0.5H, dd, J−1.2, 7.8 Hz), 4.59 (0.77H, br s, B(OH)₂ partially exchanged), 4.14 (2H, t, J=6.5 Hz), 4.04 (1H, t, J=6.5 Hz), 1.97-1.91 (2H, m), 1.90-1.83 (1H, m), 1.13 (3H, t, J=7.4 Hz), 1.08 (1.5H, t, J=7.4 Hz).

c) Preparation of 4-Amino-4′-chloro-6-isopropyl-3′-propoxy-biphenyl-3-carboxylic acid methyl ester: To a stirred mixture of 4-chloro-3-propoxybenzeneboronic acid (4.4 g, 0.021 mol), 2-amino-5-iodo-4-isopropyl-benzoic acid methyl ester (5.24 g, 0.0164 mol) and 1,1′-bis(diphenylphosphino)ferrocenedichloropalladium (II) (0.4 g, 0.49 mmol) in dry DMF (100 ml) under argon is added sodium carbonate (41 ml of a 2M aqueous solution, 0.082 mol). The mixture is heated at 80° C. for 16 h, cooled to room temperature and poured into diethyl ether (500 ml). The ether layer is separated, washed with water (3×200 ml) and then saturated brine (50 ml), dried (MgSO₄), filtered and evaporated under reduced pressure to give a brown syrup. Purification by column chromatography on silica gel using cyclohexane and then cyclohexane/ethyl acetate (50:1) as eluant followed by recrystallisation from n-hexane gave 4-Amino-4′-chloro-6-isopropyl-3′-propoxy-biphenyl-3-carboxylic acid methyl ester.

d) Preparation of 6-(4-Chloro-3-propoxy-phenyl)-7-isopropyl-2-methyl-3H-quinazolin-4-one: Hydrogen chloride gas is bubbled through a solution of 4-amino-4′-chloro-6-isopropyl-3′-propoxy-biphenyl-3-carboxylic acid methyl ester (4.9 g, 0.0136 mol) in dry acetonitrile (100 ml) for 15 min at room temperature. The bubbling is then stopped and the mixture heated at reflux for 90 min, cooled to room temperature and the volatiles removed under reduced pressure. The colourless residue is partitioned between water (500 ml) and ethyl acetate (250 ml) and sodium bicarbonate is added portion-wise until no further CO₂ evolution took place. The ethyl acetate phase is separated and washed sequentially with water (200 ml) and saturated brine (50 ml), dried (MgSO₄), filtered and evaporated under reduced pressure. The resulting colourless solid is suspended in boiling n-hexane (250 ml) and ethyl acetate (250 ml) is added until the solid dissolved. Upon cooling, this gave colourless crystalline product (6-(4-Chloro-3-propoxy-phenyl)-7-isopropyl-2-methyl-3H quinazolin-4-one) and further pure material is recovered by evaporating the mother liquor. ¹H nmr (CDCl₃, 400 MHz) 6H (ppm) 10.36 (1H, br s), 8.06 (1H, s), 7.68 (1H, s), 7.41 (1H, d, J=8.0 Hz), 6.86 (1H, d, J=1.8 Hz), 6.83 (1H, dd, J=1.8, 8.0 Hz), 4.01 (2H, t, 6.5 Hz), 3.15 (1H, m), 2.55 (3H, s), 1.91-1.85 (2H, m), 1.22 (6H, d, J=6.8 Hz), 1.08 (3H, t, J=7.4 Hz); Calc. C 68.01%, H 6.25%, N 7.55%; Found C 67.71%, H 6.00%, N 7.46%; Melting point 236° C.; HPLC RT=7.04 minutes (Phenomenex Luna C18 3 micron column (30×4.6 mm); gradient elution: 10-100% MeCN in water (+0.08% formic acid) over 10 minutes at 3.0 mL/minute); MH+371.

In the following examples compounds of formula I wherein R² is isopropyl and R³ is methyl are prepared analogously to the Examples above:

| | | | | HPLC retention |
|---|---|---|---|---|
| Example | R¹ | MS | | time [min] |
| 3 | 4-chloro-phenyl | 311.2 | M-H− | 4.93* |
| 4 | 3,5-dichloro-phenyl | 348.7 | MH+ | 5.51* |
| 5 | I | 329.1 | MH+ | 4.1* |
| 6 | 2,5-dichloro-phenyl | 347.2 | MH+ | 5.34* |
| 7 | 3-methoxy-4-chloro-phenyl | 343 | MH+ | 4.92* |
| 8 | 3-ethoxycarbonyl-4-methoxy-phenyl | 381.4 | MH+ | 4.15* |
| 9 | 3-furyl | 269.1 | MH+ | 4.27* |
| 10 | 4-chloro-3-ethoxy-phenyl | 357 | MH+ | 5.33* |
| 11 | 3-ethoxy-4-methoxy-phenyl | 352 | M+ | 4.3* |
| 12 | benzo[1,3]dioxol-5-yl | 322 | M+ | 4.3* |
| 13 | 2,2-difluorobenzo[1,3]dioxol-5-yl | 359.5 | MH+ | 5.24* |
| 14 | 3-chloro-5-methoxy-phenyl | 343.3 | MH+ | 5.11* |
| 15 | 3-chloro-5-ethoxy-phenyl | 357.2 | MH+ | 6.9* |
| 16 | 4-chloro-3-isopropoxy-phenyl | 371 | MH+ | 6.7** |
| 17 | 4-chloro-3-(2-methylpropoxy)-phenyl | 385 | MH+ | 7.5** |
| 18 | 3,5-dichloro-4methoxy-phenyl | 377 | MH+ | 6.82* |
| 19 | 2,5-dimethyl-3-furyl | 297 | MH+ | 5.3* |
| 20 | 3,5-dichloro-4-hydroxy-phenyl | 363.1 | MH+ | 5.75* |
| 21 | 2,4-dichloro-5-ethoxy-phenyl | 391.1 | MH+ | 7.1** |
| 22 | 5-methyl-isoxazol-3-yl | 284.1 | MH+ | 4** |
| 23 | 4-chloro-3-cyclopropylmethoxy-phenyl | 383 | MH+ | 6.8** |
| 24 | 4-chloro-3-fluoro-phenyl | 331 | MH+ | 5.7** |
| 25 | 4-chloro-3-(2-methoxyethoxy)-phenyl | 387 | MH+ | 5.6** |
| 26 | 4-chloro-3-butoxy-phenyl | 385 | MH+ | 7.5** |
| 27 | 4-chloro-3-(tetrahydrofuran-2- | 413 | MH+ | 6.7** |
| | ylmethoxy)-phenyl | | | |
| 28 | 4-chloro-3-(3-dimethylaminopropoxy)- | 414 | MH+ | 3.74** |
| | phenyl | | | |
| 29 | 4-chloro-3-(2,2-dimethyl)-propoxy-phenyl | 399 | MH+ | 8.14** |
| 30 | 4-chloro-3-propoxy-phenyl | 371.2 | MH+ | 7.05** |
| 31 | 4-chloro-3-(tetrahydrofuran-3- | 413 | MH+ | 6.1** |
| | ylmethoxy)-phenyl | | | |
| 32 | 4-chloro-3-(2-dimethylaminoethoxy)- | 397.3 | M+ | 3.33** |
| | phenyl | | | |
| 33 | 4-chloro-3-(3-methylbutoxy)-phenyl | 398.3 | M+ | 7.96** |
| 34 | 4-chloro-3-cyclopentoxy-phenyl | 397.2 | MH+ | 7.56** |
| 35 | 3-bromo-5-methyl-phenyl | 371 | MH+ | 6.77** |
| 36 | 4-chloro-3-(1-methylpyrrolidin-3-yloxy)- | 412.4 | MH+ | 3.66** |
| | phenyl | | | |
| 37 | 4-chloro-3-(fur-3-ylmethoxy)-phenyl | 409.2 | MH+ | 6.68** |
| 38 | 4-chloro-3-(2-methyl- | 397.2 | MH+ | 7.38** |
| | cyclopropylmethoxy)-phenyl | | | |
| 39 | 4-chloro-3-(2-isopropoxyethoxy)-phenyl | 414.4 | M+ | 6.75** |
| 40 | 4-chloro-3-(2-ethoxyethoxy)-phenyl | 400.4 | M+ | 6.34** |
| 41 | 3-chloro-4-methyl-phenyl | 327.2 | MH+ | 6.44** |
| 42 | 4-chloro-3-(2-phenethyloxy)-phenyl | 433.2 | MH+ | 7.62** |
| 43 | 4-chloro-3-[2-(2-methoxyphenyl)ethoxy]- | 463.3 | MH+ | 7.72** |
| | phenyl | | | |
| 44 | 4-chloro-3-(2-cyclopropylethoxy)-phenyl | 397.2 | MH+ | 7.53** |
| 45 | 4-chloro-3-(1-methyl-cyclopropyl- | 399.3 | M+ | 7.48** |
| | methoxy)-phenyl | | | |
| 46 | 4-chloro-3-cyclobutylmethoxy-phenyl | 397.2 | MH+ | 7.72** |
| 47 | 4-chloro-3-propylsulfanyl-phenyl | 387.2 | MH+ | 7.36** |
| 48 | 4-chloro-3-[2-(4-methoxy-phenyl)- | 463.3 | MH+ | 7.49** |
| | ethoxy]-phenyl | | | |
| 49 | 4-chloro-3-(1,1dimethyl-propoxy)-phenyl | 398.5 | M+ | 7.53** |
| 50 | 4-chloro-3-(3-fluoro-propoxy)-phenyl | 389 | MH+ | 6.54** |
| 51 | 4-chloro-3-[2-(3-methoxy-phenyl)- | 463.3 | MH+ | 7.49** |
| | ethoxy]-phenyl | | | |
| 52 | 4-chloro-3-(3-methylsulfanyl-propoxy)- | 417.2 | MH+ | 7.09** |
| | phenyl | | | |
| 53 | 4-chloro-3-methyl-phenyl | 327.2 | MH+ | 6.49** |
| 54 | 4-chloro-3-[2-(2-methoxy-ethoxy)ethoxy]- | 431.3 | MH+ | 5.82** |
| | phenyl | | | |
| 55 | 4-chloro-3-[((Z)-propenyl)oxy]-phenyl | 369 | MH+ | 6.97** |
| 56 | 4-chloro-3-(2-propoxy-ethyl)-phenyl | 399 | MH+ | 7.17** |
| 57 | 4-chloro-3-allyloxy-phenyl | 369 | MH+ | 6.62** |
| 58 | 4-chloro-3-(3-methoxy-butoxy)-phenyl | 415.2 | MH+ | 6.65** |

### EXAMPLE 59

### Preparation of 2-Amino6(4-chlorophenyl)-7-isopropyl-3H-quinazolin-4-one.

a) Preparation of 4-Amino-4′-chloro-6-isopropylbiphenyl-3-carboxylic acid: A suspension of 4-amino-4′-chloro-6-isopropylbiphenyl-3-carboxylic acid methyl ester [prepared analogously to examples above] (0.95 g, 3.13 mmol) in methanol (20 mL) under a nitrogen atmosphere was treated with 5M KOH solution (12 mL), and the mixture is heated at 80° C. for 1 h. Upon cooling to room temperature, the mixture is partitioned between ethyl acetate (50 mL) and water (100 mL) and extracted. The aqueous phase is washed with fresh ethyl acetate (50 mL). The aqueous phase is acidified to pH3 with conc. HCl solution, and extracted with ethyl acetate (2×50 mL). The combined organic layers are dried (anhydrous MgSO₄), filtered and the solvent is removed under reduced pressure to afford the crude title compound as a brown semi-solid residue. This is used without further purification, although a small sample is purified by flash chromatography (1:1 ethyl acetate-hexanes) for analytical purposes.

b) Preparation of 6-(4-Chlorophenyl)-7-isopropyl- 1H-benzo[d][1,3]oxazine-2,4-dione: A stirred suspension of 4-amino-4′-chloro-6-isopropylbiphenyl-3-carboxylic acid (0.8 g, 2.76 mmol) in anhydrous dioxane (15 mL) is treated at room temperature with trichloromethyl chloroformate (2.18 g, 11.04 mmol). The mixture is heated under reflux for 6 h. Upon cooling to room temperature, methanol (3 mL) is added and the mixture is concentrated by evaporation under reduced pressure. The resulting brown solid is recrystallized from absolute ethanol to afford the title compound as off-white crystals.
c) Preparation of 2-Amino-6-(4-chlorophenyl)-7-isopropyl-3H-quinazolin-4-one: A stirred suspension of 6-(4-chlorophenyl)-7-isopropyl-1 H-benzo[d][1,3]oxazine-2,4-dione (0.216 g, 0.68 mmol), 2-ethyl-2-thiopseudourea hydrobromide (0.126 g, 0.68 mmol) and Na₂CO₃ (0.145 g, 1.37 mmol) in MeCN (10 mL) is heated under reflux for 35 min. The condenser is removed and the bulk of the solvent is driven off. m-Xylene (6 mL) is added, the condenser is replaced and the temperature of the oil bath is raised to 150° C. A small pellet of NaOH is added, and the mixture is heated under reflux for 2.5 h. Upon cooling to room temperature, the mixture is partitioned between 0.5M NaOH solution (150 mL) and ethyl acetate (50 mL) and extracted. The aqueous phase is extracted with fresh ethyl acetate (50 mL). The combined organic layers are backwashed with brine (100 mL) and dried (anhydrous MgSO₄). The solvent is removed under reduced pressure to afford the crude title compound as an off-white solid. This is recrystallized from absolute ethanol to afford pure compound. Mp 326-330° C. ¹H nmr (DMSO-d₆, 400 MHz) δH (ppm) 10.89 (1H, s, exchanges with D₂O), 7.58 (1H, s), 7.5-7.47 (2H, dd, J=1.8, 8.4 Hz), 7.33-7.31 (2H, dd, J=1.8, 6.5 Hz), 7.18 (1H, s), 6.31 (2H, br s, exchanges with D₂O), 2.98-2.94 (1H, m), 1.13-1.12 (6H, d, J=6.8 Hz). HPLC RT=4.4 minutes (Phenomenex Luna C18 3 micron column (30×4.6 mm); gradient elution: 10-100% MeCN in water (+0.08% formic acid) over 10 minutes at 3.0 mL/minute); MH+314.06 (100%).

In the following examples compounds of formula I wherein R² is isopropyl and R³ is NH₂ are prepared analogously to the above Example 59:

| | | | |
|---|---|---|---|
| 60 | 4-chloro-3-cyclopropylmethoxy-phenyl | 384.2 MH+ | 5.08** |

In the following examples compounds of formula I wherein R² is isopropyl and R³ is methyl are prepared analogously to Examples 1 or 2:

| | | | HPLC retention |
|---|---|---|---|
| Example | R¹ | MS | time [min] |
| 61 | 4-chloro-3-(2-ethoxy-ethyl)-phenyl | 385.3 MH+ | 4.98** |
| 62 | 4-chloro-3-ethoxymethyl-phenyl | 371.3 MH+ | 4.92** |
| 63 | 4-chloro-3-(tetrahydro-furan-3-yloxy)- | 399.3 MH+ | 5.83** |
| | phenyl | | |
| 64 | 4-chloro-3-(2-hydroxy-ethoxy)-phenyl | 373.3 MH+ | 4.95** |
| 65 | 4-methoxy-3-propoxy-phenyl | 367.4 MH+ | 5.69** |
| 66 | 3-amino-4-chloro-phenyl | 328.2 MH+ | 5.23** |
| 67 | 3-butylamino-4-chloro-phenyl | 384.3 MH+ | 7.33** |
| 68 | 3,4-difluoro-5-propoxy-phenyl | 373.3 MH+ | 6.80** |
| 69 | 3,4-difluoro-5-methoxy-phenyl | 345.3 MH+ | 4.36** |
| 70 | 3-(2-chloro-ethoxy)-4,5-difluoro-phenyl | 393.2 MH+ | 6.42** |
| 71 | 3,4-difluoro-5-(3-methoxy-butoxy)-phenyl | 417.3 MH+ | 6.58** |

HPLC conditions:
*Phenomenex Kingsorb 3 micron C18 column (30 × 4.6 mm), gradient elution 10-100% MeCN in water (+0.1% TFA) over 10 minutes at 3.0 mL/min.
**Phenomenex Luna reverse phase C18 3 micron 30 × 4.6 mm; Gradient elution 10% MeCN in water (+0.08% formic acid) to 100% MeCN over 10 min (rate = 3.0 mL/min).

### EXAMPLE 72

### Soft Capsules

5000 soft gelatin capsules, each comprising as active ingredient 0.05 g of one of the compounds of formula I mentioned in the preceding Examples, are prepared as follows:

### Composition

| | | |
|---|---|---|
| Active ingredient | 250 | g |
| Lauroglycol | 2 | litres |

Preparation process: The pulverized active ingredient is suspended in Lauroglykol® (propylene glycol laurate, Gattefossé S.A., Saint Priest, France) and ground in a wet pulverizer to produce a particle size of about 1 to 3 μm. 0.419 g portions of the mixture are then introduced into soft gelatin capsules using a capsule-filling machine.

1. a quinazolinone of formula: 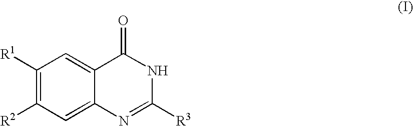
wherein
R¹ is hal; 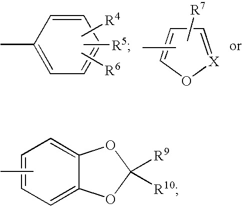
X is N or CR⁸;
R² is hal; nitro; C₁-C₈alkylcarbonyl; C₁-C₆alkyl or C₃-C₈cycloalkyl;
R³ is C₁-C₆alkyl; C₁-C₆alkoxy or amino:
R⁴ is H; hal; hydroxy; amino; C₁-C₆alkyl-amino di(C₁-C₆alkyl)-amino, C₁-C₆alkyl; C₁-C₆alkoxy which is unsubstituted or mona, di- or trisubstituted by halogen or hydroxy; C₁-C₆alkoxyC₁-C₆alkoxy; C₁C₆alkoxyC₁-C₆alkoxyC₁-C₆alkoxy; C₁-C₆alkoxyC₁-C₆alkyl; C₃-C₇cycloalkyl or C₃-C₇cycloalkylC₁-C₆alkoxy that may be substituted at the cycloalkyl residue by C₁-C₆alkyl C₁-C₆alkoxycarbonyl; C₃-C₆alkenyloxy; (C₁-C₆alkyl)₂N-C₁-C₆alkoxy; C₁-C₆alkyl-sulfanylC₁-C₈alkoxy, 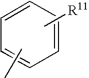
or
—O—[CH₂]ₙ-A wherein A represents 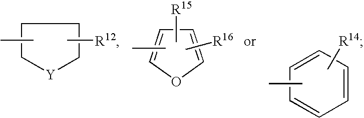
Y represents O or NR¹³,
and n is 0, 1, 2, 3, 4, 5 or 6;
R⁵ and R⁶, independently, are H; hal; C₁-C₆alkoxy; or C₁-C₆alkyl;
R⁷ and R⁸a independently are H or C₁-C₆alkyl;
R⁹ and R¹⁰, independently, are H or hal;
R¹¹ is H; hal, C₁-C₆alkoxy; or C₁-C₆alkyl
R¹² is H; hal; C₁-C₆alkoxy; or C₁-C₆alkyl;
R¹³ is H or C₁-C₆alkyl;
R¹⁴ is H; hal;: C₁-C₆alkoxy; or C₁-C₆alkyl; and
R¹⁵ and R¹⁶, independently, are H; hal; or C₁-C₆alkyl;
with the exception of the compound of formula I wherein R¹ and R² are both iodo or chloro and
R³ is methyl, and of the compound of formula I wherein R¹ and R² are both selected from fluoro and bromo and R³ is butyl,
in free base or acid addition salt form.

2. A quinazolinone of formula I according to claim 1 wherein
R¹ is hal; 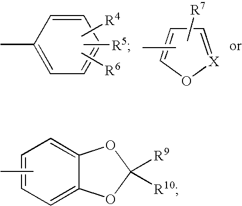
X is N or CR⁸;
R² is C₁-C₆alkyl;
R³ is C₁-C₆alkyl or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, 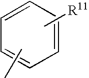
or
—O—[CH₂]ₙ-A wherein A represents 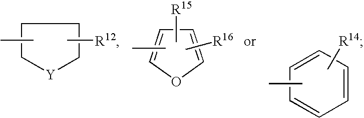
Y represents O or NR¹³,
and n is 0, 1 or 2;
R⁵ and R⁶, independently, are H; hal; or C₁-C₆alkoxy;
R⁷ and R⁸, independently, are H or C₁-C₆alkyl;
R⁹ and R¹⁰, independently, are H or hal;
R¹¹ is H; hal; C₁-C₆alkoxy; or C₁-C₆alkyl;
R¹² is H;
R¹³ is C₁-C₆alkyl;
R¹⁴ is H; or C₁-C₆alkoxy; and
R¹⁵ and R¹⁶ are H; hal; or C₁-C₆alkyl;
in free base or acid addition salt form.

3. A quinazolinone of formula I according to claim 1 wherein
R¹ is hal; 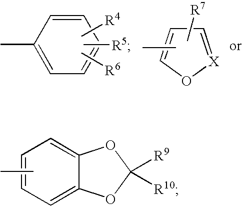
X is N or CR⁸;
R² is C₁-C₆alkyl;
R³ is C₁-C₆alkyl or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, or —O—[CH₂]ₙ-A wherein A represents 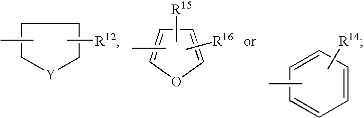
Y represents O or NR¹³,
and n is 0, 1 or 2;
R⁵ and R⁶, independently, are H; hal; or C₁-C₆alkoxy;
R⁷ and R⁸, independently, are H or C₁-C₆alkyl;
R⁹ and R¹⁰, independently, are H or hal;
R¹² is H;
R¹³ is C₁-C₆alkyl;
R¹⁴ is H; or C₁-C₆alkoxy; and
R¹⁵ and R¹⁶ are H; hal; or C₁-C₆alkyl;
in free base or acid addition salt form.

4. A compound of formula I according to claim 1 wherein
R¹ is hal; 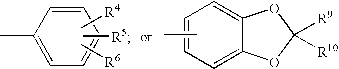
R² is hal; nitro; C₁-C₆alkylcarbonyl; C₁-C₆alkyl or C₃-C₆cycloalkyl;
R³ is C₁-C₆alkyl; C₁-C₆alkoxy or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, 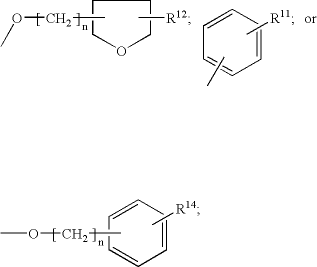
wherein n is 0, 1, 2, 3, 4, 5 or 6;
R⁵, R⁶, R¹¹ and R¹⁴, independently, are H hal; C₁-C₆alkoxy; or C₁-C₆alkyl;
R¹² is H or C₁-C₆alkyl; and
R⁹ and R¹⁰, independently are H or hal;
with the exception of the compound of formula I wherein R¹ and R² are both iodo or chloro and R³ is methyl, and of the compound of formula I wherein R¹ and R² are both selected from fluoro and bromo and R³ is butyl,
in free base or acid addition salt form.

5. A compound of formula II 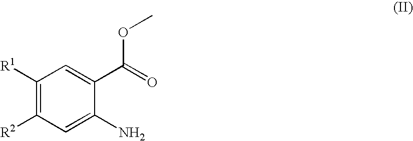
wherein
R¹ is hal; 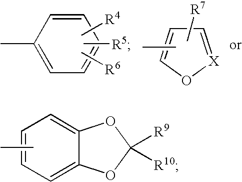
and
R² is hal; nitro; C₁-C₆alkylcarbonyl; C₁-C₆alkyl or C₃-C₆cycloalkyl.

6. A process for the preparation of a compound of formula I 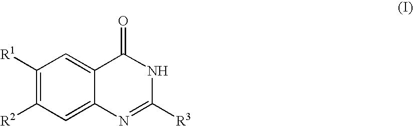
wherein
R¹ is hal; 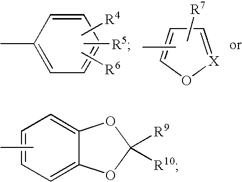
X is N or CR⁸;
R² is hal; nitro; C₁C₆alkylcarbonyl; C₁-C₆alkyl or C₃-C₈cycloalkyl;
R³ is C₁-C₆alkoxy or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, 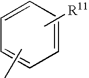
or
—O—[CH₂]ₙ-A wherein A represents 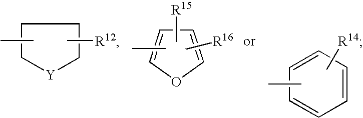
Y represents O or NR¹³,
and n is 0, 1 or 2;
R⁵ and R⁶, independently, are H; hal; or C₁-C₆alkoxy;
R⁷ and R⁸, independently, are H or C₁-C₆alkyl;
R⁹ and R¹⁰, independently, are H or hal;
R¹¹ is H; hal; C₁-C₆alkoxy; or C₁-C₆alkyl;
R¹² is H;
R¹³ is C₁-C₆alkyl;
R¹⁴ is H; or C₁-C₆alkoxy; or C₁-C₆alkyl; and
R¹⁵ and R¹⁶ independently, are H; hal; or C₁-C₆alkyl; with the exception of the compound of formula I where R¹ and R² are both selected from fluor and bromo and R³ is butyl,
or a salt thereof, comprising the step of
a) for the production of a compound of formula I wherein R³ is not NH₂, reacting a compound of formula II 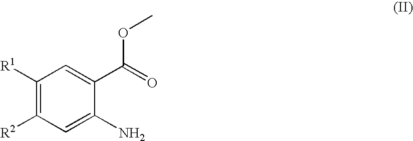
wherein
R¹ and R² are as defined above, with a compound of formula III
N≡C—R³ (III)
wherein R³ is as defined in claim 1; or
b) for the production of a compound of formula I wherein R³ is NH₂, reacting a compound of formula IV 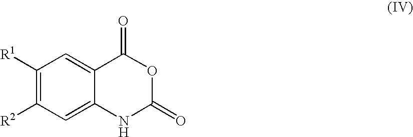
wherein R¹ and R² is as defined above,
with 2-ethyl-2-thiopseudourea hydrobromide;
and recovering the obtained compound, in free or in salt form.

7. (canceled)

8. (canceled)

9. A pharmaceutical composition comprising a compound of claim 1 in free base or pharmaceutically acceptable acid addition salt form, in association with a pharmaceutical carrier or diluent.

10. (canceled)

11. (canceled)

12. A method for treating or preventing a disease or condition in which vanilloid receptor activation plays a role or is implicated comprising administering to a mammal in need thereof a therapeutically effective amount of a quinazolinone of formula I 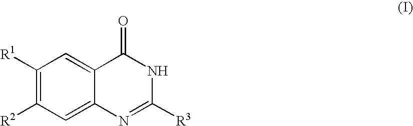
wherein
R¹ is hal; 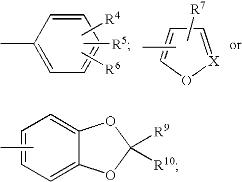
X is N or CR⁸;
R² is hal; nitro; C₁-C₆alkylcarbonyl; C₁-C₆alkyl or C₃-C₈cycloalkyl;
R³ is C₁-C₆alky; C₁-C₆alkoxy or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, 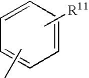
or
—O—[CH₂]ₙ-A wherein A represents 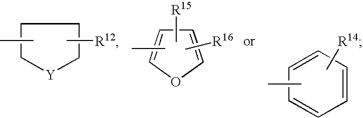
Y represents O or NR¹³,
and n is 0, 1 or 2;
R⁵ and R⁶, independently, are H; hal; or C₁-C₆alkoxy;
R⁷ and R⁸, independently, are H or C₁-C₆alkyl;
R⁹ and R¹⁰, independently, are H or hal;
R¹¹ is H; hal; C₁-C₆alkoxy; or C₁-C₆alkyl;
R¹² is H; hal; C₁-C₆alkoxy; or C₁-C₆alkyl;
R¹³ is C₁-C₆alkyl;
R¹⁴ is H; or C₁-C₆alkoxy; and
R¹⁵ and R¹⁶, independently, are H; hal; or C₁-C₆alkyl;
in free base or acid addition salt form.

13. A pharmaceutical composition for the treatment or prevention of a diseases or condition in which vanillold receptor activation plays a role or is implicated comprising a quinazolinone of formula I 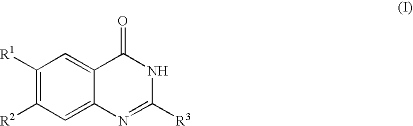
wherein
R¹ is hal; 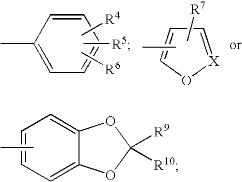
X is N or CR⁸;
R² is hal; nitro; C₁-C₆alkylcarbonyl; C₁-C₆alky or C₃-C₆cycloalkyl;
R³ is C₁-C₆alkyl; C₁-C₆alkoxy or amino;
R⁴ is hal; hydroxy; amino; C₁-C6alkyl-amino, C₁-C₆alkyl; C₁-C₆alkoxy which is unsubstituted or monosubstituted by halogen or hydroxy; C₁-C₆alkoxyC₁-C₆alkoxy; C₁-C₆alkoxyC₁-C₆alkoxyC₁-C₆alkoxy; C₁-C₆alkoxyC₁-C₆alkyl; C₃-C7cycloalkyl or C₃-C₇cycloalkylC₁-C₆alkoxy that may be substituted at the cycloalkyl residue by C₁-C₆alkyl; C₁-C₆alkoxycarbonyl; C₃-C₆alkenyloxy; (C₁-C₆alkyl)₂N-C₁-C₆alkoxy; C₁-C₆alkyl-sulfanyl; C₁-C₆alkyl-sulfanylC₁-C₆alkoxy, 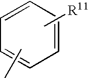
or
—O—[CH₂]ₙ-A wherein A represents 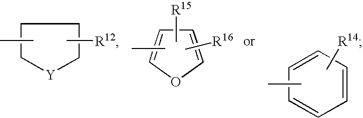
Y represents O or NR¹³,
and n is 0, 1 or 2;
R⁵ and R⁶, independently, are H; hal; or C₁-C₆alkoxy; or C₁-C₆alkyl;
R⁷ and R⁸, independently, are H or C₁-C₆alkyl;
R⁹ and R¹⁰, independently, are H or hal;
R¹¹ is H; hal; C₁-C₆alkoxy; or C₁-C₆alkyl;
R¹² is H; C₁-C₆alkoxy; or C₁-C₆alkyl;
R¹³ is C₁-C₆alkyl;
R¹⁴ is H; or C₁-C₆alkoxy; or C₁-C₆alkyl; and
R¹⁵ and R¹⁶, independently, are H; hal; or C₁-C₆alkyl;
and a carrier.
